(19) United States
(12) Patent Application Publication
(10) Pub. No.: US 2022/0117673 A1
(43) Pub. Date: Apr. 21, 2022

(54) EndoInspect Apparatus, System and Method

(71) Applicants: Arthur C. McKinley, Westport, MA (US); Jesse R. Plouffe, Westport, MA (US); John H. Westgate, Westport, MA (US); Jamie Korte, Westport, MA (US); David W. Sanso, Golden, CO (US)

(72) Inventors: Arthur C. McKinley, Westport, MA (US); Jesse R. Plouffe, Westport, MA (US); John H. Westgate, Westport, MA (US); Jamie Korte, Westport, MA (US); David W. Sanso, Golden, CO (US)

(21) Appl. No.: 17/503,491

(22) Filed: Oct. 18, 2021

Related U.S. Application Data
(60) Provisional application No. 63/093,453, filed on Oct. 19, 2020.

(51) Int. Cl. A61B 34/20; A61B 1/00; A61B 1/06

(52) U.S. Cl. CPC A61B 34/20; A61B 1/00006; A61B 1/0684; A61B 2034/2059; A61B 1/0016; A61B 1/00105; A61B 1/00009; A61B 1/00036

(57) ABSTRACT

Advances in endoscope inspection systems to provide for evidence of the cleanliness of critical portions of the instrument's interior. Utilizing borescope sensing technology, the precise location of the borescope within the endoscope can be repeatedly established is in order to establish the healthy condition of the endoscope. Light intensity of cleaning paraphernalia may be adjusted to prevent endoscope damage.

### CROSS-REFERENCE TO RELATED APPLICATIONS

This application claims priority to co-pending U.S. Provisional patent applications Ser. No. 63/093,453 titled “EndoProcess Apparatus, System and Method”, filed on Oct. 19, 2020, the disclosure of which are herein incorporated by reference in their entirety.

### PATENTS CITED

The following documents and references are incorporated by reference in their entirety, Stephenson (U.S. Pat. No. 10,709,313) and Cheong (WO 2020/096892).

### FIELD OF THE INVENTION

The invention is about inspection of small cavities, and specifically about an apparatus, system and method to ensure that cavities within endoscopes and surgical tools are checked for reliable operation in combined endoscopic systems.

### DESCRIPTION OF THE RELATED ART

In recent times, a veritable cornucopia of endoscopic and laparoscopic surgical tools has resulted in an amalgamation of fiberscopes, videoscopes, illuminators and other assorted surgical devices becoming available for clinicians and doctors.

However, if the lumens or other interior cavities of these endoscopes is not appropriately clean when re-used, hospital acquired infections may be induced. To ensure safety, these devices must be reliably cleaned and checked for cleanliness and integrity between each patient.

To accomplish this, a borescope inspection system is introduced to inspect and record the interior of the cleaned inner cavities. What is needed, is a set of mechanical, electronic and optical tools to unify the process of assuring adequate system function.

### SUMMARY OF THE INVENTION

This section is for the purpose of summarizing some aspects of the present invention and to briefly introduce some embodiments. Simplifications or omissions may be made to avoid obscuring the purpose of the section. Such simplifications or omissions are not intended to limit the scope of the present invention.

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e., that it will be taken to mean an inclusion of not only the listed components it directly references, but also other non-specified components or elements. This rationale will also be used when the term ‘comprised’ or ‘comprising’ is used in relation to one or more steps in a method or process.

In one aspect, the invention is about an elongated component travel position indicator comprising: a mechanical travel encoding assembly that mechanically connects to an endoscope and tracks the passage through said assembly of an elongated component into said endoscope's lumen interior and wherein said encoding assembly can be left mechanically attached to said endoscope during disinfection. In another aspect said elongated mechanical components may be comprised of one or more of the following: optical borescopes, digital borescopes, brushes, fiberoptics transmission light and/or LED lights. In yet another aspect said elongated mechanical component displacement is detected via one or more of the following: image capture analysis displacement calculation, rotating components, elongated component surface mark detection and/or elongated component RFID detection. In another aspect said position indicator automatically detects the type of elongated mechanical component whose displacement is being measured.

In one aspect, the invention is about a control unit capable of interfacing with one or more types of borescope camera systems, including the capability to recognize said borescope type and set display and control parameters, a display and human control interface. In another aspect touch screen control on display screen for all system functions, lighting control of said borescope, image capture control, keyboard for annotation of images and image interface for recording of images and annotations. In yet another aspect one or more external cameras with lighting and optics suitable to inspect all outer surfaces. In another aspect an elongated component travel position indicator comprising a mechanical travel encoding assembly that mechanically connects to an endoscope and tracks the passage through said assembly of an elongated component into said endoscope's lumen interior and wherein said encoding assembly can be left mechanically attached to said endoscope during disinfection. In yet another aspect said elongated mechanical components may be comprised of one or more of the following: optical borescopes, digital borescopes, brushes, fiberoptics transmission light and/or LED lights.

In another aspect said elongated mechanical component displacement is detected via one or more of the following: image capture analysis displacement calculation, rotating components, elongated component surface mark detection and/or elongated component RFID detection. In yet another aspect said position indicator automatically detects the type of elongated mechanical component whose displacement is being measured. In another aspect said control unit has the ability to control the power being fed to any LED assembly connected to said unit and if said control unit senses a static image being fed from any elongated component within said endoscope lumen for more than a preset time period, the power being fed to any LED inside said lumen is reduced to a lower level until motion is sensed again from said image. In yet another aspect the light source used for imaging is comprised of switchable light sources of a frequency suitable for optical biological diagnostics.

In another aspect, the invention is about a system for the automated feeding of a borescope into an endoscope lumen, said system comprising a device that positions the borescope properly for a given endoscope and feeds the borescope into the lumen correctly, a manual or motorized feed, said feed under system control to run through a known path length; and indicators to show position of borescope and position of images captured along the path. In another aspect an automatic system brake that would not allow movement while diagnostic lighting is on.

In another aspect, the invention is about a system for the prevention of damage to an endoscope lumen, said system comprising: a control unit that has the ability to control the power being fed to any LED assembly connected to said unit, an image analysis component capable of detecting that the image from said assembly within said lumen has been static for a preset period of time; and wherein said control unit reduces the level of power to said LED assembly within preset safe levels until motion is sensed again from said image and/or an operator over-ride signal is received.

Other features and advantages of the present invention will become apparent upon examining the following detailed description of an embodiment thereof, taken in conjunction with the attached drawings, which are provided for purposes of illustration and not of limitation.

### BRIEF DESCRIPTION OF THE DRAWINGS

FIG. 1 illustrates a borescope going into the body of an endoscope, according to an exemplary embodiment of the invention.

FIG. 2 illustrates an endoscope inspection system, according to an exemplary embodiment of the invention.

FIG. 3 illustrates a fiberoptic emitting light to sterilize lumen using germicidal wavelengths borescope tool, according to an exemplary embodiment of the invention.

FIG. 4 illustrates an LED emitting light to sterilize lumen using germicidal wavelengths borescope tool, according to an exemplary embodiment of the invention.

FIG. 5 illustrates a standalone position encoder for a borescope, according to an exemplary embodiment of the invention.

FIG. 6 illustrates a liquid or air channel built into a borescope for cleaning or clearing the lumens, according to an exemplary embodiment of the invention.

FIG. 7 illustrates a system diagram for a mechanical borescope feeder that positions the borescope properly for a given endoscope and feeds the borescope into the lumens, according to an exemplary embodiment of the invention.

The above-described and other features will be appreciated and understood by those skilled in the art from the following detailed description and drawings.

### DETAILED DESCRIPTION OF THE INVENTION

To provide an overall understanding of the invention, certain illustrative embodiments and examples will now be described. However, it will be understood by one of ordinary skill in the art that the same or equivalent functions and sequences may be accomplished by different embodiments that are also intended to be encompassed within the spirit and scope of the disclosure. The compositions, apparatuses, systems and/or methods described herein may be adapted and modified as is appropriate for the application being addressed and that those described herein may be employed in other suitable applications, and that such other additions and modifications will not depart from the scope hereof.

As used in the specification and claims, the singular forms “a”, “an” and “the” include plural references unless the context clearly dictates otherwise. For example, the term “a transaction” may include a plurality of transaction unless the context clearly dictates otherwise. As used in the specification and claims, singular names or types referenced include variations within the family of said name unless the context clearly dictates otherwise.

Certain terminology is used in the following description for convenience only and is not limiting. The words “lower,” “upper,” “bottom,” “top,” “front,” “back,” “left,” “right” and “sides” designate directions in the drawings to which reference is made, but are not limiting with respect to the orientation in which the modules or any assembly of them may be used.

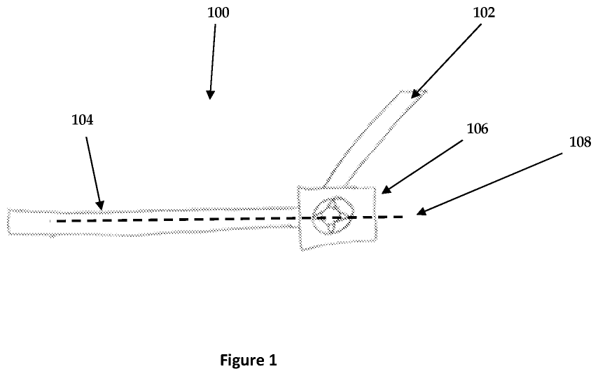

Referring to FIG. 1, we see a typical endoscope 100 as used by many surgeons. The unit 100 typically has a handle 102 (typically designed to remain outside the human body) and one or more hollow portions 102 (sometimes called a lumen/tube) that is designed to go into the human body. To allow for access to the body cavity, the back of the unit 106 typically has a closable opening to allow access through the opening 104, or there is a path via the handle 102.

Over time, when the unit 100 is reused, there have been documented times where the cleaning of the cavities is not thorough, leaving biological material in all or parts of the cavities within 102/104. To correct for this situation, hospital maintenance departments have begun to inspect and clean these cavities through the insertion of a narrower elongated mechanical component 108 (e.g. borescope, brush, optical fiber). When inspecting, the elongated mechanical component may be an optical or electronic borescope.

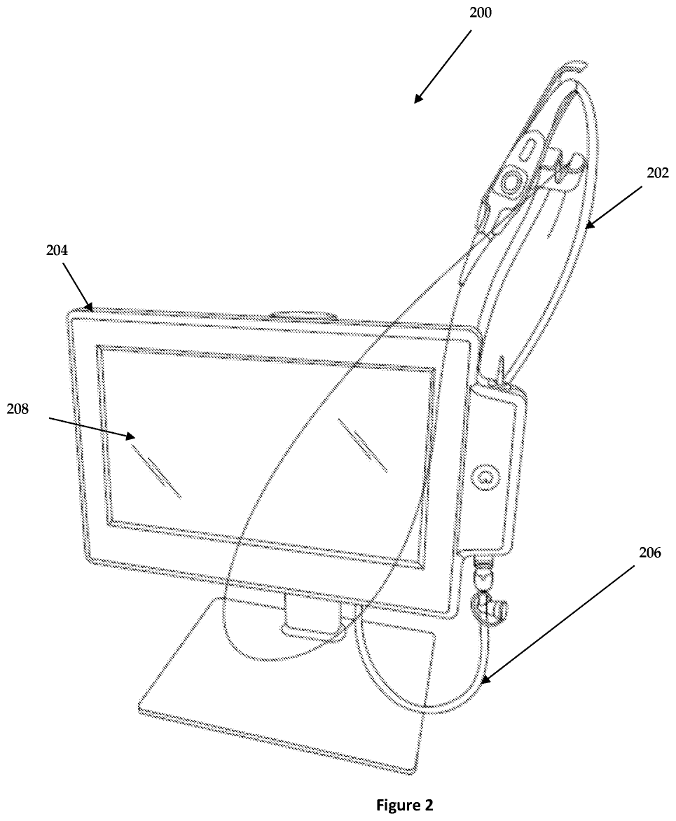

One proposed solution 200, can be seen when looking at FIG. 2, where a borescope 108 (in this case equipped with a video link) is connected via an interface cable 202 to a control unit 204, from which the magnification/brightness of any light source can be controlled by the operator. The video feed 206 is shown 208 to the operator and potentially recorded.

The challenge of using a high magnification narrow view optical device like a borescope (optical or video) 108 in/out of a long endoscope lumen 104 is that upon discovering that a biological matter, crud or other anomaly is present along the tube 104, one must be capable of quickly revisiting that area, i.e. to go back quickly and reliably to the location. The present way to do it, is for the technician to mark the depth at which the borescope 108 detected the failure with their finger.

In one embodiment, the system 200 could be programmed to recognize borescope type and set display and control parameters to suit. This would be easily accomplished via electronic interfacing with the device, by reading the device 1-D/2-D barcode tag or RFID tag, or simply by having a system take an image and confirm the identity of the unit.

In one embodiment, the system 200 could be equipped with a Touch screen control on display screen for all system functions, including controls for lighting, image capture, keyboard and/or voice tagging and annotation of the captured images.

In one embodiment, a valuable feature of the control unit 206, would be an Auto LED 402 intensity control, so as to reduce both the heat emission of the electronics, as well as any potential energy delivery to a surface regulatory limitation. In one embodiment, the LED is built into a plastic housing with limited cooling and operates at full power in certain modes and automatic reduced power modulation in other modes.

In another embodiment, a reference image library may be added in a seamless fashion to the unit screens and the image capture functions. In certain cases, images may only be stored in local physical media (such as USB FOB drives, similar devices or secure link encrypted drives) so that captured image confidentiality may be maintained. In cases where a procedure may be similar to another already performed, reference images stored on internal memory may be used by the operator for clarification of flaws or product appearance when normal. In such cases, updates may be issued when new products are being inspected or brought to market.

In another embodiment, a needed tool is an External camera with lighting connected to the control unit 204 in order to inspect outer surfaces, so that one or more cameras may be used for procedure inspection, including before and after procedure documentation. Similarly, a magnifying/zoom glass or lens may be mounted to such a system, to allow inspection of the endoscope and ancillary tools outer surfaces, including built-in lighting, and light source filters.

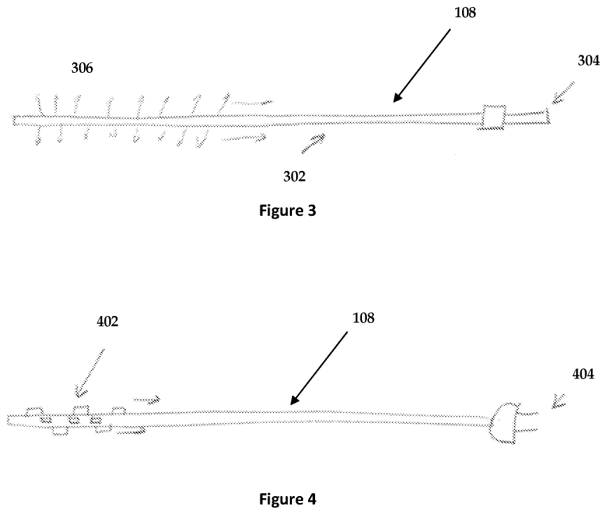

Additional ways to sterilize the interior of the lumens 104 may include the insertion of a lighting system that can enhance identification of biological matter (by targeting the frequency at which the matter is highly visible) may be used. In one such case (FIG. 3), a light for visible use is built into a borescope 108 using fiberoptics 302 fed from a light source (Incandescent or LED 304) in the handle section that come out at one or more points 306 along the length of the unit 108. Such fibers can be switched to a different light source frequency that enhances detection of contamination, cancel cells, etc.

In a similar vein, switchable lighting may change from broadband visible light to specific light temperature/frequency diagnostic lights. As an added bonus, in one embodiment, the proposed Auto borescope feeder would not allow movement while diagnostic lighting is on, preventing accidents or blurry images. In another embodiment (FIG. 4), this may include a lighting device borescope 108 to sterilize the lumen using germicidal wavelengths. A string of LEDs 402 or fiberoptic bundle emitting germicidal light can be passed into lumen in order to sterilize the lumen after it has been cleaned, all connected to the outside 404 for power.

To appreciate an example of such an operation, to eliminate the heat effects from the LED 402, in one embodiment, if the camera control components within the control unit 204 senses a static image for a preset time period, the power to the LED is reduced to a level that can be sustained by cooling capacity indefinitely. As soon as motion is again detected, the LED resumes its automatic mode.

A key component of the invention is the ability to measure the position of the borescope 108 within the lumen 104 of the endoscope 100. This may be accomplished through a number of ways. One is to analyze each image captured by the camera on the borescope 108 as it travels along the body of the lumens 104, and calculate the displacement by analyzing the displacement of notable features within said image. This image capture analysis displacement calculation will provide the distance traveled within the body of the lumens.

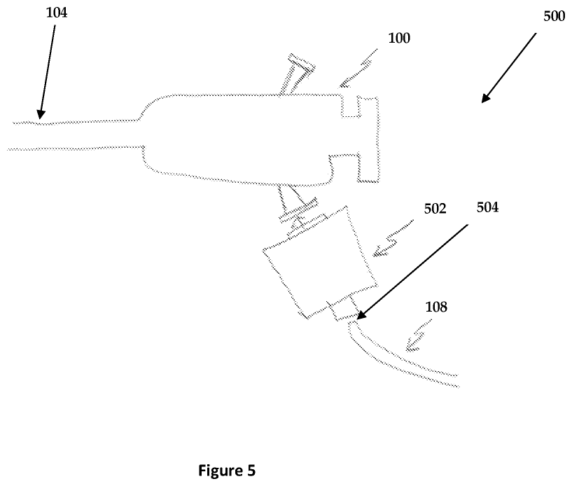

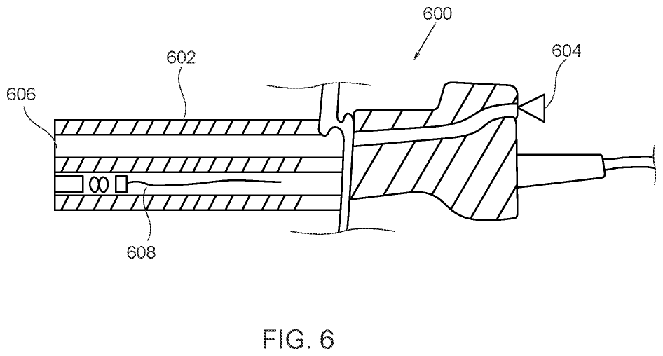

In another embodiment 500 (FIG. 5), a separate borescope 108 and borescope tip 504 travel and position indicator is comprised of a separate borescope/elongated component displacement/travel/encoder unit 502 that potentially identifies the type of component being used to go through it and the endoscope lumens (e.g. optical borescopes, video borescopes, brush, small pipe). Said ID may be manually input to the control unit 206 via the operator, and/or automatically via AutoID (barcode, 2D symbology, RFID scanning). The unit 502 uses a variety of mechanical components to determine and tracks the position of the borescope within the endoscope lumens 104 as the borescope is inserted and retracted.

Besides an approach as described above (a series of images processed by the control unit), the encoder unit 502 may work in a variety of ways. In one embodiment, one or more wheels within said encoder 502 are displaced and rotated by the insertion of the elongated member (e.g. endoscope 108, brush, air tube) as it travels in/out of the scope 100. In another embodiment, similar to an optical mouse, the outer diameter of the borescope has marks that are read as the shaft goes by, so that the one or more regular marks optical marks along the body of the borescope 108 are read by an optical sensor or other sensors within the encoder 502, to determine the distance. Similarly, a series of RFID tags along the length of the borescope 108 allow for a similar distance sensing that is much more robust to crud on the shaft.

The encoder 502 interfaces with the control unit 206 in order to couple the information with the video. This information may be displayed by the control unit 206 to show the position of the borescope tip 504 within the scope (in a computer display), as well as used as ancillary data when recording the position of biologicals and other features in images captured along the endoscope 100 internal path.

Another improvement is a stand-alone position encoder for a borescope. In one embodiment, this is a device that the borescope passes through and that may be attached to a port on an endoscope lumen. The encoder would read the depth of the borescope (as it passes through), so that the borescope may be quickly returned to a previous position after a defect has been found or otherwise necessitated the removal from the lumen. Such an encoder would also be used to read the position of brushes and tools so that a specific area can be cleaned. Such an encoder may be built so as to be left on the endoscope during disinfection.

As an improvement to the above, the image display/capture systems may include multiple switchable inputs. In one embodiment, different sizes/types of borescopes or cameras can remain connected and ready for use with a switching control to select one for display.

In many cases, the lumens becomes clogged during a procedure, for which we propose a system 600 having pneumatic and/or hydraulic systems 604 to clean and clear lumens 104. Such a system would connect 606 to the endoscope channel 104, having air/gases and liquid tight connectors. Then using either air, gas, cleaning media or cleaning chemicals in any combination the lumens could be cleared of any partial or complete obstructions. The endoscope 608 optics would be protected.

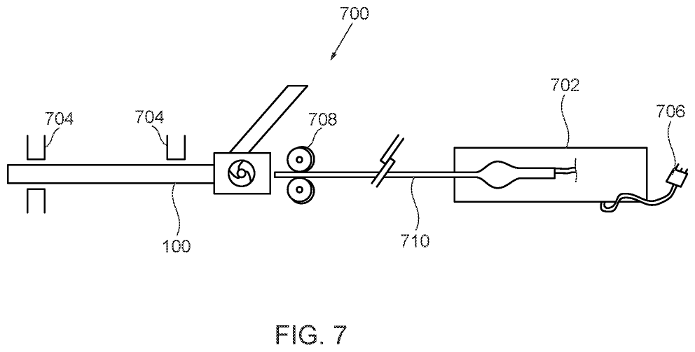

Referring to FIG. 7, we see another proposed improvement, a Mechanical borescope feeder 700. In one embodiment, a device 702 controls the displacement position of the borescope 710 properly (along one or more rollers 708) for a given endoscope 100 and feeds the borescope into the lumen, guided by a distance encoder 706. One or more fixtures 704 holds the endoscope 100.

The feeding may be manual, or through an optional motorized feed. In one embodiment, the feed 702 can be manually controlled or programed to run automatically through a known path length. Any deviation from known/expected motor force may be used to slow/alert the operator, reducing errors caused by unexpected obstructions. In addition, when any anomaly is detected by the system, the borescope position may be frozen and/or registered for later review.

In a similar vein, in one embodiment, air/gases or liquid channels could be built into borescope for cleaning or clearing lumens. Such a channel could then be used to clear debris or dry the lumen as visual inspection is taking place.

### Conclusion

In concluding the detailed description, it should be noted that it would be obvious to those skilled in the art that many variations and modifications can be made to the shown embodiments without substantially departing from the principles of the present invention. Also, such variations and modifications are intended to be included herein within the scope of the present invention as set forth in the disclosure.

It should be emphasized that the above-described embodiments of the present invention, particularly any “exemplary embodiments” are merely possible examples of the implementations, merely set forth for a clear understanding of the principles of the invention. Any variations and modifications may be made to the above-described embodiments of the invention without departing substantially from the spirit of the principles of the invention. All such modifications and variations are intended to be included herein within the scope of the disclosure and present invention.

The present invention has been described in sufficient detail with a certain degree of particularity. The utilities thereof are appreciated by those skilled in the art. It is understood to those skilled in the art that the present disclosure of embodiments has been made by way of examples only and that numerous changes in the arrangement and combination of parts may be resorted without departing from the spirit and scope of the invention.

1. An elongated component travel position indicator comprising:
a mechanical travel encoding assembly that mechanically connects to an endoscope and tracks the passage through said assembly of an elongated component into said endoscope's lumen interior; and
wherein said encoding assembly can be left mechanically attached to said endoscope during disinfection.

2. the position indicator of claim 1 wherein:
said elongated mechanical components may be comprised of one or more of the following:
optical borescopes, digital borescopes, brushes, fiberoptics transmission light and/or LED lights.

3. the position indicator of claim 2 wherein:
said elongated mechanical component displacement is detected via one or more of the following:
image capture analysis displacement calculation, rotating components, elongated component surface mark detection and/or elongated component RFID detection.

4. the position indicator of claim 3 wherein:
said position indicator automatically detects the type of elongated mechanical component whose displacement is being measured.

5. An endoscope cavity inspection system comprising:
a control unit capable of interfacing with one or more types of borescope camera systems, including the capability to recognize said borescope type and set display and control parameters;
a display; and
human control interface.

6. the system of claim 5 further comprising:
touch screen control on display screen for all system functions;
lighting control of said borescope;
image capture control;
keyboard for annotation of images; and
image interface for recording of images and annotations.

7. the system of claim 6 further comprising:
one or more external cameras with lighting and optics suitable to inspect all outer surfaces.

8. the system of claim 5 further comprising:
an elongated component travel position indicator comprising a mechanical travel encoding assembly that mechanically connects to an endoscope and tracks the passage through said assembly of an elongated component into said endoscope's lumen interior; and
wherein said encoding assembly can be left mechanically attached to said endoscope during disinfection.

9. the system of claim 8 wherein:
said elongated mechanical components may be comprised of one or more of the following:
optical borescopes, digital borescopes, brushes, fiberoptics transmission light and/or LED lights.

10. the system of claim 9 wherein:
said elongated mechanical component displacement is detected via one or more of the following:
image capture analysis displacement calculation, rotating components, elongated component surface mark detection and/or elongated component RFID detection.

11. the system of claim 10 wherein:
said position indicator automatically detects the type of elongated mechanical component whose displacement is being measured.

12. The system of claim 5 wherein:
said control unit has the ability to control the power being fed to any LED assembly connected to said unit; and
if said control unit senses a static image being fed from any elongated component within said endoscope lumen for more than a preset time period, the power being fed to any LED inside said lumen is reduced to a lower level until motion is sensed again from said image.

13. The system of claim 5 wherein:
the light source used for imaging is comprised of switchable light sources of a frequency suitable for optical biological diagnostics.

14. A system for the automated feeding of a borescope into an endoscope lumen, said system comprising:
a device that positions the borescope properly for a given endoscope and feeds the borescope into the lumen correctly;
a manual or motorized feed, said feed under system control to run through a known path length; and
indicators to show position of borescope and position of images captured along the path.

15. the system of claim 14 further comprising:
an automatic system brake that would not allow movement while diagnostic lighting is on.

16. A system for the prevention of damage to an endoscope lumen, said system comprising:
a control unit that has the ability to control the power being fed to any LED assembly connected to said unit;
an image analysis component capable of detecting that the image from said assembly within said lumen has been static for a preset period of time; and
wherein said control unit reduces the level of power to said LED assembly within preset safe levels until motion is sensed again from said image and/or an operator over-ride signal is received.
